(19) United States
(12) Patent Application Publication
(10) Pub. No.: US 2023/0088890 A1
(43) Pub. Date: Mar. 23, 2023

(54) PORTABLE STERILIZER FOR PERSONAL PROTECTIVE EQUIPMENT

(71) Applicant: KWJ Engineering, Inc., Newark, CA (US)

(72) Inventor: Joseph R. Stetter, Hayward, CA (US)

(21) Appl. No.: 17/933,993

(22) Filed: Sep. 21, 2022

Related U.S. Application Data
(60) Provisional application No. 63/246,377, filed on Sep. 21, 2021.

(51) Int. Cl. A61L 2/20; A61L 2/26

(52) U.S. Cl. CPC A61L 2/202; A61L 2/26; A61L 2202/122; A61L 2202/121; A61L 2202/123; A61L 2202/14; A61L 2202/15

(57) ABSTRACT

A sterilization system that includes a sterilization tank having a closed end and an open end and a lid assembly having a chamber facing side opposite an outward facing side. The lid assembly is removably engageable with the sterilization tank at the open end of the sterilization tank thereby forming a sterilization chamber when engaged with the sterilization tank. The sterilization system also includes one or more ozone sources and one or more fans coupled to the chamber facing side of the lid assembly such that the one or more ozone sources and the one or more fans are disposed in the sterilization chamber when the lid assembly is engaged with the sterilization tank and a controller communicatively coupled to the one or more ozone sources and the one or more fans.

### CROSS-REFERENCE TO RELATED APPLICATIONS

This application claims benefit of U.S. provisional patent application Ser. No. 63/246,377 entitled “PORTABLE STERILIZER FOR PERSONAL PROTECTIVE EQUIPMENT” filed Sep. 21, 2021, which is incorporated herein by reference in its entirety.

### TECHNICAL FIELD

The present specification generally relates to sterilization systems, and more particularly, to sterilization systems designed to sterilize personal protective equipment using ozone.

### BACKGROUND

The rapid spread of the COVID-19 pandemic worldwide has taxed the availability of N95 masks and other personal protective equipment. Widespread ability to sterilize and re-use N95 masks will help relieve this critical issue in the short-term. It is also clear that additional long-term benefits of sterilization and re-use will include lowering operating cost, improving material supply chain logistics, and reducing waste.

Accordingly, a need exists for inexpensive and portable sterilization systems for sterilization of personal protective equipment.

### SUMMARY

In one embodiment, a sterilization system includes a sterilization tank having a closed end and an open end and a lid assembly having a chamber facing side opposite an outward facing side. The lid assembly is removably engageable with the sterilization tank at the open end of the sterilization tank thereby forming a sterilization chamber when engaged with the sterilization tank. The sterilization system also includes one or more ozone sources and one or more fans coupled to the chamber facing side of the lid assembly such that the one or more ozone sources and the one or more fans are disposed in the sterilization chamber when the lid assembly is engaged with the sterilization tank and a controller communicatively coupled to the one or more ozone sources and the one or more fans.

In another embodiment, a method of disinfecting a specimen includes generating ozone in a sterilization chamber of a sterilization system using one or more ozone sources disposed in the sterilization chamber. The sterilization system includes a sterilization tank having a closed end and an open end and a lid assembly having a chamber facing side opposite an outward facing side. The lid assembly is engaged with the sterilization tank at the open end of the sterilization tank thereby forming the sterilization chamber. The one or more ozone sources and one or more fans are coupled to the chamber facing side of the lid assembly, a controller is communicatively coupled to the one or more ozone sources, and the specimen is disposed in the sterilization chamber of the sterilization system. The method further includes circulating ozone in the sterilization chamber using the one or more fans thereby disinfecting the specimen disposed in the sterilization chamber.

In yet another embodiment, a sterilization system includes a sterilization tank having a closed end and an open end and a lid assembly including an interior mounting substrate coupled to an exterior mounting substrate. The lid assembly is removably engageable with the sterilization tank at the open end of the sterilization tank thereby forming a sterilization chamber when engaged with the sterilization tank. The interior mounting substrate includes an interior mounting surface at a chamber facing side of the lid assembly and the exterior mounting substrate includes an exterior mounting surface at the outward facing side of the lid assembly. The lid assembly further includes an intermediate coupling substrate disposed between and coupled to both the interior mounting surface and the exterior mounting substrate. The intermediate coupling substrate comprises an engagement region configured to engage with the sterilization tank. The sterilization system further includes one or more ozone sources and one or more fans coupled to the interior mounting surface of the interior mounting substrate such that the one or more ozone sources and the one or more fans coupled are disposed in the sterilization chamber when the lid assembly is engaged with the sterilization tank and a controller and a power supply coupled to the exterior mounting surface of the exterior mounting substrate. The controller is communicatively coupled to the one or more ozone sources and the power supply is electrically coupled to the controller and the one or more ozone sources.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A schematically depicts a sterilization system comprising a sterilization chamber and a multilayer lid assembly, according to one or more embodiments shown and described herein;

FIG. 1B schematically depicts a sterilization system comprising a sterilization chamber and a single layer lid assembly, according to one or more embodiments shown and described herein;

FIG. 2 schematically depicts an exploded view of the sterilization system with the lid assembly of FIG. 1A, according to one or more embodiments shown and described herein;

FIG. 3 schematically depicts an example embodiment of the sterilization system of FIGS. 1A and 2, according to one or more embodiments shown and described herein;

FIG. 4 schematically depicts an interior mounting substrate of the lid assembly of the sterilization system of FIGS. 1A and 2 having fans and ozone sensors mounted thereon, according to one or more embodiments shown and described herein;

FIG. 5 schematically depicts an ozone sensor positioned on an ozone sensor mount, according to one or more embodiments shown and described herein;

FIG. 6 schematically depicts the interior mounting substrate of FIG. 4 coupled to an intermediate coupling substrate, according to one or more embodiments shown and described herein;

FIG. 7 schematically depicts an exterior mounting substrate of the lid assembly of the sterilization system of FIGS. 1A and 2, according to one or more embodiments shown and described herein;

FIG. 8 schematically depicts the exterior mounting substrate of FIG. 7, further including a display mounted thereon, according to one or more embodiments described herein;

FIG. 9 schematically the interior mounting substrate of FIG. 5, further including a pump assembly mounted thereon, according to one or more embodiments shown and described herein; and

FIG. 10 graphically depicts the levels of bacteriophage present on specimen's that have and have not undergone sterilization process using the sterilization system of the present disclosure, according to one or more embodiments shown and described herein;

### DETAILED DESCRIPTION

Embodiments described herein are directed to a sterilization system that includes a sterilization tank and a lid assembly that is removably engageable with an open end of the sterilization tank to form a sterilization chamber. The sterilization system is designed to sterilize specimens, such as personal protective equipment, disposed in the sterilization chamber using ozone (i.e., O₃). The lid assembly comprises a chamber facing side opposite an outward facing side and the sterilization system further includes ozone sources and fans coupled to the chamber facing side of the lid assembly such that the when the lid assembly is engaged with the sterilization tank the ozone sources and fans are disposed in the sterilization chamber. Control unit components, such as a controller and a power supply for operating and powering the ozone sources and fans may also be coupled to the lid assembly, for example, the outward facing side of the lid assembly. In some embodiments, the lid assembly is formed by multiple substrates coupled together, such as an interior mounting substrate that provides a mounting surface for the ozone sources, ozone sensors, and fans, and an exterior mounting substrate that provides a mounting surface for the control unit components. In other embodiments, the lid assembly comprises a single, shared mounting surface substrate. In operation, ozone may be generated by the ozone sources and circulated in the sterilization chamber by the fans to sterilize a specimen, such as personal protective equipment, positioned in the sterilization chamber.

The sterilization system of the present disclosure is an inexpensive and portable modular sterilizer design which requires no toxic chemical consumables and generates ozone on demand as needed in precisely controlled concentrations and cycle times. The sterilization system is inexpensive and portable enough to provide sterilization of PPE to all types of healthcare facilities, clinics and emergency response teams, regardless of size or location. Additionally, the sterilization system is capable of sterilizing N95 and medical masks, which will allow other non-emergency care users to sterilize and reuse masks and PPE rather than disposing of them. This will reduce the demand, allowing more ready access to new PPE for emergency healthcare workers and reduce waste and cost. Various embodiments of the sterilization system and methods of sterilizing a specimen using the sterilization system are described in more detail herein. Whenever possible, the same reference numerals will be used throughout the drawings to refer to the same or like parts.

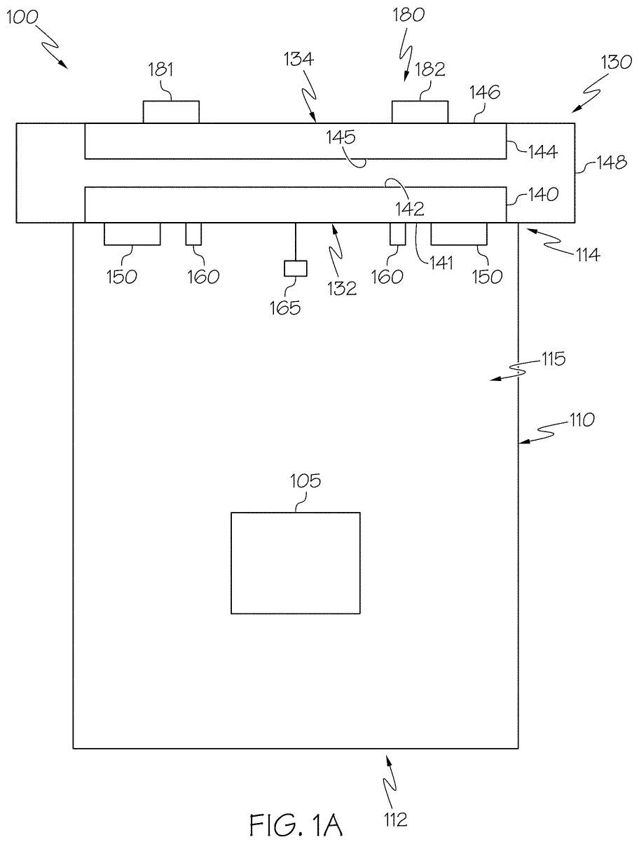

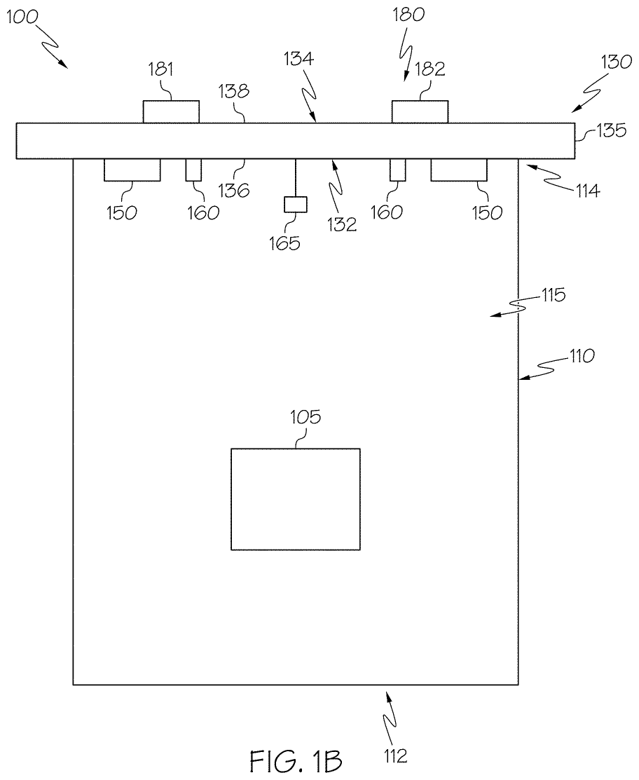

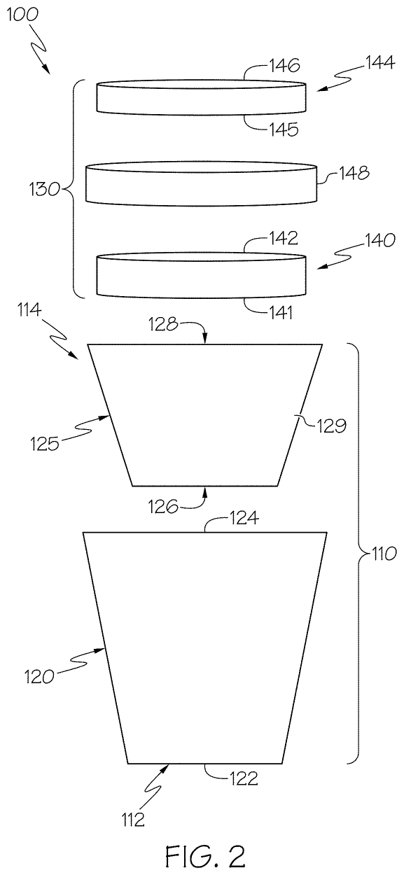

Referring now to FIGS. 1A-2 a sterilization system 100 comprising a sterilization tank 110 and a lid assembly 130 is depicted. The sterilization tank 110 comprises a closed end 112 opposite an open end 114. The lid assembly 130 comprises a chamber facing side 132 opposite an outward facing side 134. The lid assembly 130 is removably engageable with the sterilization tank 110 at the open end 114 of the sterilization tank 110 thereby forming a sterilization chamber 115 when engaged with the sterilization tank 110. The sterilization system 100 comprises one or more ozone sources 160 and one or more fans 150 coupled to the chamber facing side 132 of the lid assembly 130 such that the one or more ozone sources 160 and the one or more fans 150 are disposed in the sterilization chamber 115 when the lid assembly is engaged with the sterilization tank 110. In some embodiments, the one or more ozone sources 160 comprise a Ozotech Poseidon 200 ®, but it should be understood that other ozone sources may be contemplated.

The sterilization system 100 further comprises one or more sensors, including at least one or more ozone sensors 165 configured to measure the concentration of ozone in the sterilization chamber 115. In some embodiments, the one or more ozone sensors 165 may comprise printed ozone sensors. For example, the one or more ozone sensors 165 may comprise DGSDK sensors from SPEC Sensors, LLC of Newark, Calif. The one or more ozone sensors 165 are disposed in the sterilization chamber 115 when the lid assembly 130 is engaged with the sterilization tank 110. Additional sensors include relative humidity sensors and temperature sensors which, like the one or more ozone sensors 165, may be disposed in the sterilization chamber 115 when the lid assembly 130 is engaged with the sterilization tank 110. For example, the sensors may be coupled to the sterilization tank 110, coupled to the chamber facing side 132 of the lid assembly 130, suspended in the sterilization chamber 115 (e.g., hanging by a wire or other mounting device), or combinations thereof.

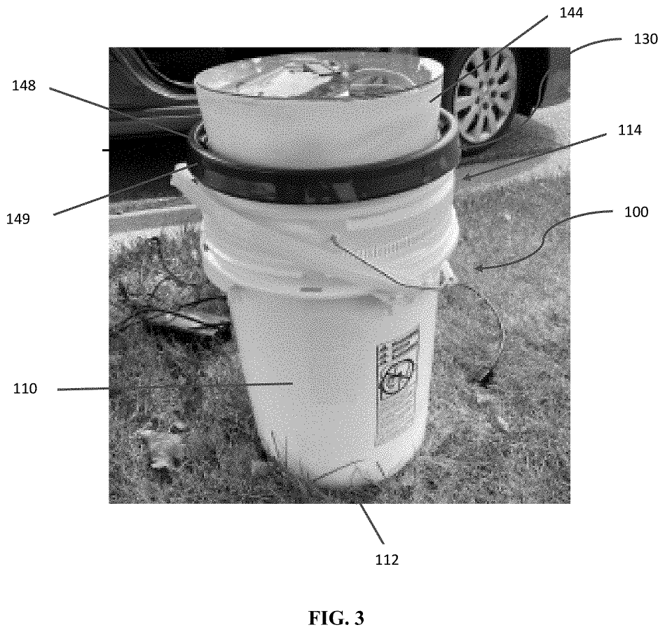

Referring now to FIG. 1A, in some embodiments, the lid assembly 130 comprises a multilayer assembly. In particular, the lid assembly 130 may comprise an interior mounting substrate 140 coupled to an exterior mounting substrate 144, the interior mounting substrate 140 comprising an interior mounting surface 141 at the chamber facing side 132 of the lid assembly 130 and the exterior mounting substrate 144 comprises an exterior mounting surface 146 at the outward facing side 134 of the lid assembly 130. In some embodiments, the exterior mounting substrate 144 and the interior mounting substrate 140 may be directly coupled such that a top surface 142 of the interior mounting substrate 140 contacts a bottom surface 145 of the exterior mounting substrate 144. In other embodiments, as depicted in FIGS. 1A and 2, the lid assembly 130 further comprises an intermediate coupling substrate 148 disposed between and coupled to the interior mounting substrate 140 and the exterior mounting substrate 144. The intermediate coupling substrate 148 comprises an engagement region 149 (FIG. 3) configured to engage with the sterilization tank 110 thereby engaging the lid assembly 130 with the sterilization tank 110.

Referring now to FIG. 1B, in some embodiments, the lid assembly 130 comprises a single layer assembly. In particular, the lid assembly 130 may comprise a shared mounting substrate 135 having an interior surface 136 at the chamber facing side 132 of the lid assembly 130 and an exterior surface 138, opposite the interior surface 136 and disposed at the outward facing side 134 of the lid assembly 130. In some embodiments, as shown in FIG. 1B, the one or more ozone sources 160 and the one or more fans 150 are coupled to the interior surface 136 of the shared mounting substrate 135 and the controller is coupled to the exterior mounting surface of the shared mounting substrate 135.

Referring now to FIG. 2, an exploded view of the sterilization system 100 with the lid assembly of FIG. 1A is depicted. As shown in FIG. 2, in some embodiments, the sterilization tank 110 comprises an inner shell 125 disposed in an outer shell 120. The outer shell 120 comprises a base 122 forming the closed end 112 of the sterilization tank 110 and an outer opening 124 opposite the base 122. The inner shell 125 comprises an inner bottom opening 126 and an inner top opening 128. The outer opening 124 of the outer shell 120 and the inner top opening 128 of the inner shell 125 collectively form the open end 114 of the sterilization tank 110. The inner shell 125 also comprises an upper perimeter 129 surrounding the inner top opening 128. In some embodiments, the upper perimeter 129 is engageable with the engagement region 149 of the intermediate coupling substrate 148 of the lid assembly 130. In other embodiments, the upper perimeter 129 is engageable with the shared mounting substrate 135 of the lid assembly 130 of FIG. 1B.

Referring now to FIG. 3, in some embodiments, the lid assembly 130 and the sterilization tank 110 are formed using two buckets, for example, two five gallon plastic buckets. In particular, the outer shell 120 may be a first five gallon plastic bucket and the inner shell 125 may be a second five gallon bucket that has its bottom cut off to form the inner bottom opening 126. In this two bucket design, the cut off bottom portion of the inner shell 125 may be used to form the exterior mounting substrate 144 of the lid assembly 130. In some embodiments, the sterilization system 100 may also comprise an outer cover engageable with the outer shell 120 of the sterilization tank 110 to enclose the lid assembly 130 within the sterilization tank 110. This may be useful when transporting the sterilization system 100.

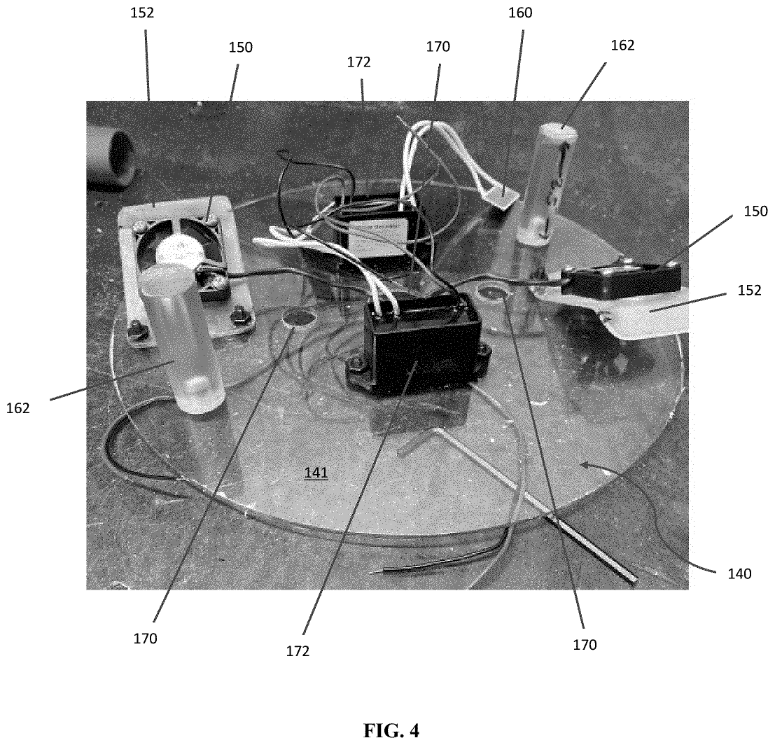

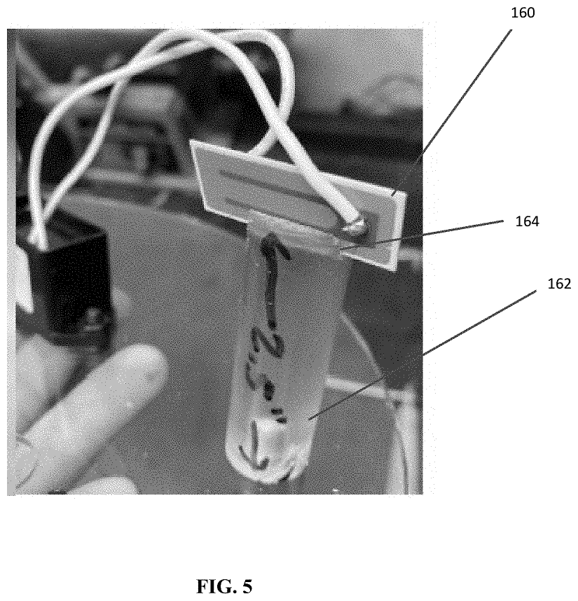

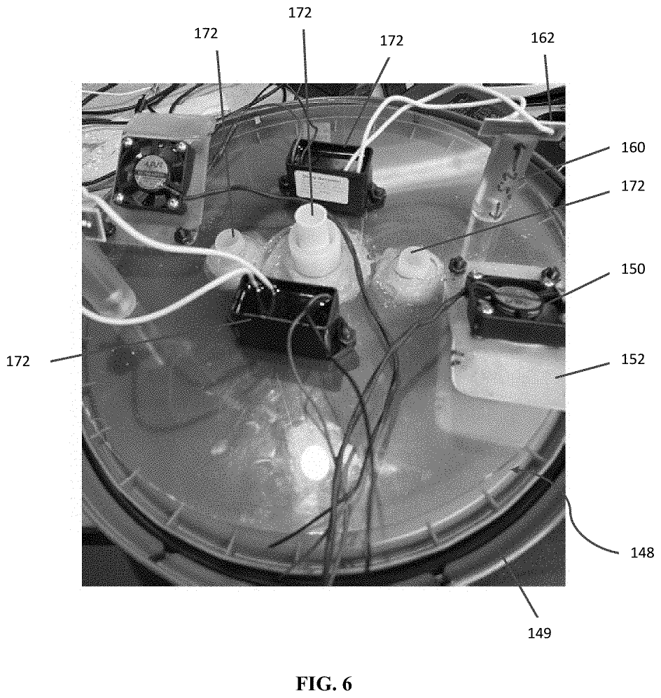

Referring now to FIG. 4-6, the interior mounting substrate 140 of the lid assembly 130 is depicted. FIG. 4 depicts fans 150, bulkhead fittings 172, and ozone source holders 162 mounted on the interior mounting surface 141 of the interior mounting substrate 140. FIG. 5 depicts an ozone source 160 mounted on an ozone source holder 162, and FIG. 6 depicts the interior mounting substrate 140 coupled to the intermediate coupling substrate 148. In some embodiments, the interior mounting substrate 140 comprises polycarbonate. As shown in FIGS. 4 and 5, the one or more ozone source holders 162 couple the one or more ozone sources 160 to the interior mounting substrate 140 (i.e., to the chamber facing side 132 of the lid assembly 130) while spacing the one or more ozone sources 160 away from the interior mounting substrate 140 (i.e., away from the chamber facing side 132 of the lid assembly 130). The one or more ozone source holders 162 may comprise a slot 164 sized to hold an ozone source 160. The slot 164 may be particularly useful in embodiments in which the ozone sources 160 are thin, printed sensors. The ozone source holders 162 may be a polycarbonate rod, however other materials and shapes are contemplated.

Referring now to FIGS. 4 and 6, in some embodiments the one or more fans 150 may be coupled to the interior mounting substrate 140 (i.e., the chamber facing side 132 of the lid assembly 130) using one or more fan mounts 152. In particular, the one or more fan mounts 152 couple the interior mounting substrate 140 at an angle relative to the interior mounting substrate 140 such that the one or more fans 150 are non-parallel with the interior mounting substrate 140, for example, a 30° angle. Indeed, the angle of the fan mounts 152 (and thus the fans 150) may correspond with the length of the ozone source holders 162 and the relative positioning of the fans 150 and the ozone source holders 162 such that the fans 150 are directed at the ozone sources 160 coupled to the end of the ozone source holders 162. In some embodiments, the sterilization system 100 comprises the same number of fans 150 and fan mounts 152 as ozone sources 160 and ozone source holders 162 such that each ozone source 160 has a fan 150 directed toward it. Moreover, each of the fans 150 may be positioned at radially outward and positioned facing in either both a clockwise direction or both a counter clockwise direction to generate a circulating airflow within the sterilization chamber 115 to circulate ozone during a sterilization process.

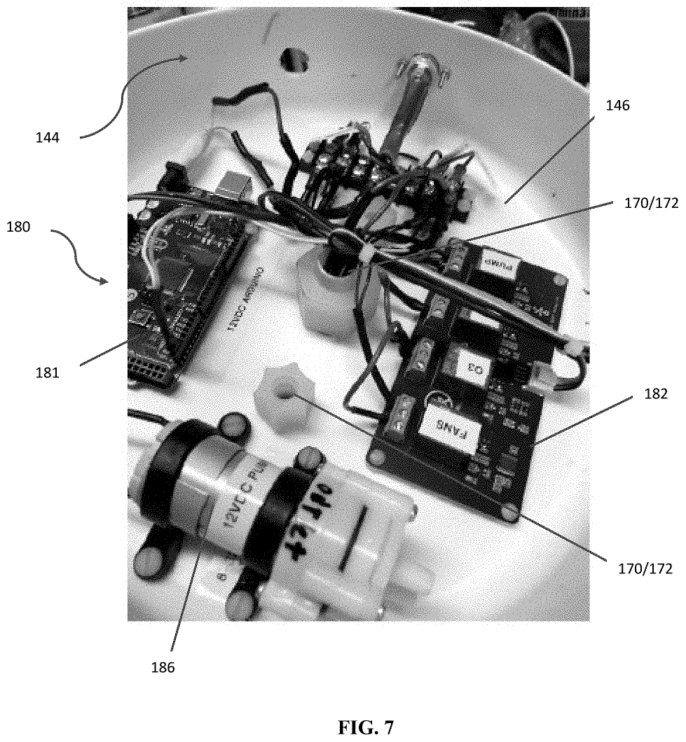

Referring again to FIGS. 4-6, the interior mounting substrate 140 comprises a plurality of mounting holes 170 and at least one bulkhead fitting 172 disposed in at least one of the plurality of mounting holes 170. As shown in FIGS. 4-6, the plurality of mounting holes 170 and the corresponding bulkhead fittings 172 may be positioned radially inward the fans 150 and the ozone sources 160. The bulkhead fittings 172 provide locations for wiring to extend through the interior mounting substrate 140 to couple the fans 150, ozone sources 160, and sensors (such as ozone sensors 165) with additional components of the sterilization system 100, such as a controller 181 and a power supply 182. The bulkhead fittings 172 may also provide locations for tubing to extend into the interior mounting substrate 140 to fluidly couple the sterilization chamber 115 with a pump assembly 186 (FIGS. 7-9). Example material that may be used to form the bulkhead fittings 172 include Kynar®, polypropylene, polyethylene.

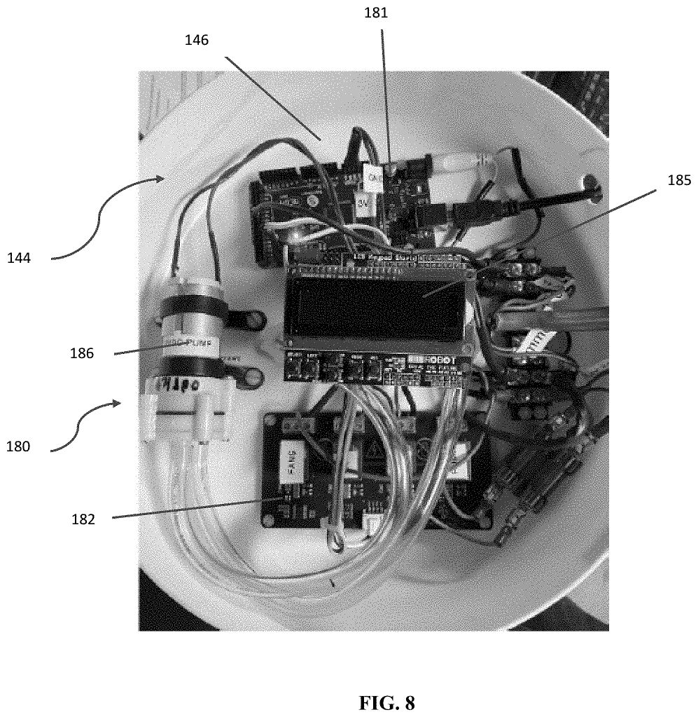

Referring now to FIGS. 7 and 8, the exterior mounting substrate 144 of the lid assembly 130 is depicted. The exterior mounting substrate 144 provides a mounting surface (e.g., the exterior mounting surface 146) for the control unit components 180 of the sterilization system 100, such as the controller 181, the power supply 182, a relay shield 183, a data storage shield 184, and as shown in FIG. 8, a display 185. In operation, the display 185 may display information regarding conditions in the sterilization chamber 115, such as ozone concentration, temperature, and relative humidity. The display 185 may also provide a report at the end of a sterilization cycle. The controller 181 is communicatively coupled to the one or more fans 150, the ozone sources 160, the sensors, including the ozone sensors 165, and may provide control signals to each. Indeed, the controller 181 may instruct the ozone sources to generate different amounts of ozone based on sensor signals received by the ozone sensors 165. The controller 181 is also communicatively coupled to the relay shield 183, the data storage shield 184, and the display 185 to provide control instructions to each. Moreover, the power supply 182 may be communicatively coupled to each component to the control unit components 180, as well as the fans 150, ozone sources 160, and sensors, providing power to each. In some embodiments, an ozone sensor may be positioned on the exterior mounting surface 146 of the exterior mounting substrate 144 and communicatively coupled to the controller 181. This external ozone sensor may be used to determine whether ozone is leaking from the sterilization chamber 115. In operation, if the external ozone sensor measures an ozone leak, the controller 181 may instruct the ozone sources 160 to stop generating ozone.

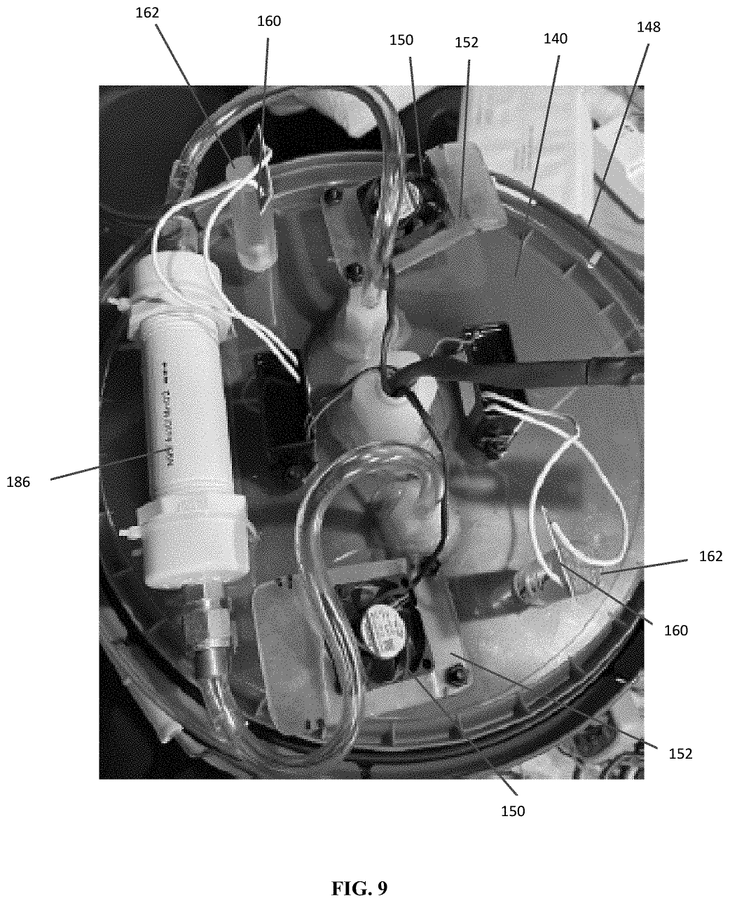

Referring still to FIGS. 7 and 8, the control unit components 180 further comprising a pump assembly 186 and a filter each fluidly coupled to the sterilization chamber 115 when the lid assembly 130 is engaged with the sterilization tank 110. The filter is positioned such that fluid (e.g., ozone and other gases) removed from the sterilization tank 110 by the pump assembly 186 traverses the filter. In some embodiments, the filter is an ozone destruct filter. In operation, the ozone destruct filter destroys ozone removed from the sterilization chamber 115 after completing a sterilization cycle and before opening the lid assembly 130. Moreover, FIG. 9 shows that embodiments are contemplated in which the pump assembly 186 is mounted to the interior mounting substrate 140. One example pump assembly 186 is a Whisper AP300®.

Referring now to FIGS. 1A-9, the sterilization system 100 may be used to disinfect a specimen 105, such as an item of personal protective equipment. This process includes placing one or more specimens 105 in the sterilization tank 110 and then securing the lid assembly 130 to the sterilization tank 110 to form and enclose the sterilization chamber 115. When multiple specimens 105 (e.g., multiple pieces of personal protective equipment) are placed in the sterilization tank 110, an insert, such as a mask rack, may be used to separate each specimen 105 to allow space for air flow. Once the specimen 105 is in the sterilization tank 110 and the sterilization chamber 115 is enclosed, ozone is generated in the sterilization chamber 115 using the one or more ozone sources 160 and the ozone is circulated in the sterilization chamber 115 using the one or more fans 150 thereby disinfecting the specimen 105 disposed in the sterilization chamber 115. In some embodiments, the specimen 105 may be sterilized in an hour or less. Thus, the sterilization system 100 may be support multiple field operations, providing repeated sterilization of personal protective equipment.

In one example experiment to measure the effectiveness of ozone treatment using the sterilization system 100, specimens 105 comprising samples of the TLS bacteriophage on cellulose disks (Remel® filter paper discs), N95 mask material (VFlex® from 3M), and vinyl sheets (Fisherbrand® microscope cover slips) underwent a sterilization process in the sterilization chamber 115. Each piece was 0.5 cm². The TLS bacteriophage was diluted to 10⁹ plaque-forming units (PFU)/ml. 2.5 microliters of TLS dilution was added to the mask material and 25 microliters of a 10⁸PFU/ml TLS dilution was added to the filter disks and the vinyl sheets. Sterilization processes were performed at 300 ppm ozone for 15 minutes, 30 minutes, and 60 minutes. After treatment in the sterilization chamber 115, specimens 105 were removed, placed in lysogeny broth (LB broth), and shaken for 30 seconds. Serial dilutions (1:10) of these samples were then prepared. A 100 microliter sample of each sample dilution, 250 microliters of overnight-grown E. coli culture in LB broth, and four milliliters of top agar were mixed in a sterile culture tube and spread on an LB agar plate. Plates were incubated overnight at 37° C. TLS plaques were counted the following day to determine the results.

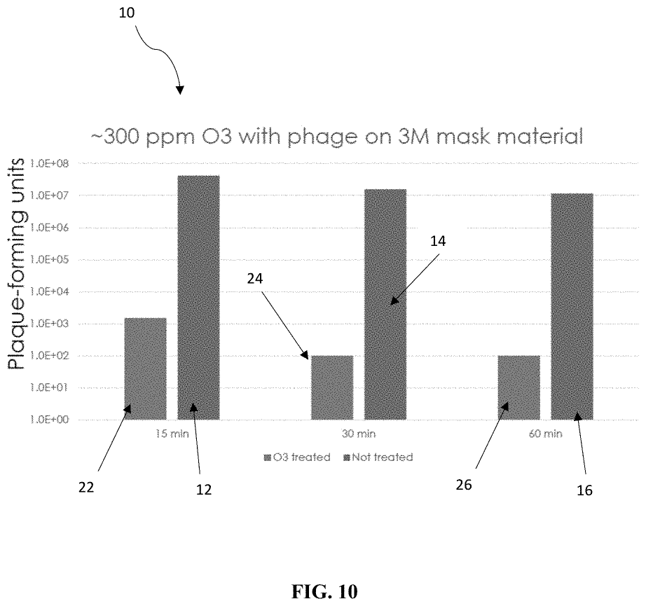

Referring now to FIG. 10, graph 10 of FIG. 10 shows the resultant amount of PFU/ml TLS on a mask material using this example experiment with 2.5 microliters of TLS dilution that is untreated after 15 minutes (bar 12), 30 minutes (bar 14) and 60 minutes (bar 16) and treated in the sterilization chamber 115 at 300 ppm ozone after 15 minutes (bar 22), 30 minutes (bar 24) and 60 minutes (bar 26). As shown by graph 10, a substantial amount of the TLS bacteriophage is removed by the sterilization process in the sterilization chamber 115.

Using the example experiment, wet samples of TLS bacteriophage on cellulose disks, FFP3 N95 mask material, or on vinyl sheets showed a 2-3 log reduction in active bacteriophage when treated with greater than 200 ppm ozone and ambient relative humidity (45-55%) but less than one log reduction in activity if the bacteriophage sample is dried on the material before ozone treatment. However, when performing the example experiment with close to 100% relative humidity in the sterilization chamber 115, there is a greater than 4-5 log reduction in active bacteriophage within one hour, regardless of material and whether the initial sample was wet or dried. This demonstrates that the sterilization system 100 is capable of inactivating TLS bacteriophage as designed and, in high relative humidity conditions, an increase in TLS inactivation (e.g., sterilization) is observed. For example, at a 25 ppm concentration of ozone in with 80% relative humidity or greater, there is a 2 log reduction in TLS activity, at 50 ppm ozone there is a 3 log reduction, at 100 ppm there is a 3-4 log reduction, and at 200 ppm ozone there is a 4-5 log reduction.

It is noted that the term “substantially” may be utilized herein to represent the inherent degree of uncertainty that may be attributed to any quantitative comparison, value, measurement, or other representation. This term is also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A sterilization system comprising:
a sterilization tank comprising a closed end and an open end;
a lid assembly comprising a chamber facing side opposite an outward facing side, wherein the lid assembly is removably engageable with the sterilization tank at the open end of the sterilization tank thereby forming a sterilization chamber when engaged with the sterilization tank;
one or more ozone sources and one or more fans coupled to the chamber facing side of the lid assembly such that the one or more ozone sources and the one or more fans are disposed in the sterilization chamber when the lid assembly is engaged with the sterilization tank; and
and a controller communicatively coupled to the one or more ozone sources and the one or more fans.

2. The sterilization system of claim 1, further comprising one or more ozone sensors disposed in the sterilization chamber when the lid assembly is engaged with the sterilization tank, wherein the one or more ozone sensors are communicatively coupled to the controller.

3. The sterilization system of claim 1, further comprising one or more ozone source holders coupling the one or more ozone sources to the chamber facing side of the lid assembly while spacing the one or more ozone sources away from the chamber facing side of the lid assembly.

4. The sterilization system of claim 3, wherein the one or more ozone source holders each comprise a polycarbonate rod.

5. The sterilization system of claim 1, further comprising one or more fan mounts coupling the one or more fans to the chamber facing side of the lid assembly at an angle relative to the chamber facing side of the lid assembly such that the one or more fans are non-parallel with the chamber facing side of the lid assembly.

6. The sterilization system of claim 1, wherein the lid assembly comprises an interior mounting substrate coupled to an exterior mounting substrate, the interior mounting substrate comprising an interior mounting surface at the chamber facing side of the lid assembly and the exterior mounting substrate comprises an exterior mounting surface at the outward facing side of the lid assembly.

7. The sterilization system of claim 6, wherein the interior mounting substrate comprise a plurality of mounting holes and at least one bulkhead fitting disposed in at least one of the plurality of mounting holes.

8. The sterilization system of claim 6, wherein the interior mounting substrate comprises polycarbonate.

9. The sterilization system of claim 6, wherein the lid assembly further comprises an intermediate coupling substrate disposed between and coupled to both the interior mounting surface and the exterior mounting substrate.

10. The sterilization system of claim 6, wherein the intermediate coupling substrate comprises an engagement region configured to engage with the sterilization tank.

11. The sterilization system of claim 1, wherein the lid assembly comprises a shared mounting substrate having an interior mounting surface opposite an exterior mounting surface, wherein the one or more ozone sources and the one or more fans are coupled to the interior mounting surface of the shared mounting substrate and the controller is coupled to the exterior mounting surface of the shared mounting substrate.

12. The sterilization system of claim 1, wherein the sterilization tank comprises a plastic material.

13. The sterilization system of claim 1, wherein the controller is coupled to the outward facing side of the lid assembly.

14. The sterilization system of claim 1, further comprising a power supply and a display coupled to the outward facing side of the lid assembly and communicatively coupled to the controller.

15. The sterilization system of claim 1, further comprising a relay shield and a data storage shield each coupled to the outward facing side of the lid assembly and communicatively coupled to the controller.

16. The sterilization system of claim 1, further comprising a filter and a pump assembly coupled to the outward facing side of the lid assembly, wherein when the lid assembly is engaged with the sterilization tank the pump assembly is fluidly coupled to the sterilization tank and the filter is positioned such that fluid removed from the sterilization tank by the pump assembly traverses the filter.

17. A method of disinfecting a specimen, the method comprising:
generating ozone in a sterilization chamber of a sterilization system using one or more ozone sources disposed in the sterilization chamber, wherein
the sterilization system comprises:
a sterilization tank comprising closed end and an open end;
a lid assembly comprising a chamber facing side opposite an outward facing side, wherein the lid assembly is engaged with the sterilization tank at the open end of the sterilization tank thereby forming the sterilization chamber;
the one or more ozone sources and one or more fans coupled to the chamber facing side of the lid assembly; and
a controller communicatively coupled to the one or more ozone sources; and
the specimen is disposed in the sterilization chamber of the sterilization system; and
circulating ozone in the sterilization chamber using the one or more fans thereby disinfecting the specimen disposed in the sterilization chamber.

18. The method of claim 17, wherein the specimen comprises personal protective equipment.

19. A sterilization system comprising:
a sterilization tank comprising closed end and an open end;
a lid assembly comprising an interior mounting substrate coupled to an exterior mounting substrate, wherein:
the lid assembly is removably engageable with the sterilization tank at the open end of the sterilization tank thereby forming a sterilization chamber when engaged with the sterilization tank;
the interior mounting substrate comprising an interior mounting surface at a chamber facing side of the lid assembly;
the exterior mounting substrate comprises an exterior mounting surface at the outward facing side of the lid assembly; and
an intermediate coupling substrate disposed between and coupled to both the interior mounting surface and the exterior mounting substrate, wherein the intermediate coupling substrate comprises an engagement region configured to engage with the sterilization tank;
one or more ozone sources and one or more fans coupled to the interior mounting surface of the interior mounting substrate such that the one or more ozone sources and the one or more fans coupled are disposed in the sterilization chamber when the lid assembly is engaged with the sterilization tank; and
a controller and a power supply coupled to the exterior mounting surface of the exterior mounting substrate, wherein the controller is communicatively coupled to the one or more ozone sources and the power supply is electrically coupled to the controller and the one or more ozone sources.

20. The sterilization system of claim 19, further comprising one or more ozone sensors communicatively coupled to the controller and disposed in the sterilization chamber when the lid assembly is engaged with the sterilization tank.

21. The sterilization system of claim 19, wherein:
the sterilization tank comprises an inner shell disposed in an outer shell;
the outer shell comprise a base forming the closed end of the sterilization tank and an outer opening opposite the base;
the inner shell comprises an inner bottom opening and an inner top opening;
the outer opening of the outer shell and the inner top opening of the inner shell collectively form the open end of the sterilization tank.

22. The sterilization system of claim 21, wherein the inner shell comprises an upper perimeter surrounding the inner top opening and engageable with the engagement region of the intermediate coupling substrate of the lid assembly.

23. The sterilization system of claim 21, further comprising an outer cover engageable with the outer shell of the sterilization tank to enclose the lid assembly within the sterilization tank.
